(19) United States
(12) Patent Application Publication
(10) Pub. No.: US 2024/0415744 A1
(43) Pub. Date: Dec. 19, 2024

(54) HAIR CONDITIONER COMPOSITIONS

(71) Applicant: THOS. BENTLEY & SON LIMITED, Leeds, West Yorkshire (GB)

(72) Inventor: John Michael STORY, Leeds, West Yorkshire (GB)

(21) Appl. No.: 18/698,506

(22) PCT Filed: Oct. 6, 2022
(86) PCT No.: PCT/GB2022/052531

§ 371 Date: Apr. 4, 2024

(30) Foreign Application Priority Data
Oct. 6, 2021 (GB) 2114319.3

(51) Int. Cl. A61K 8/02; A61K 8/36; A61K 8/41; A61K 8/44; A61K 8/73; A61K 8/92; A61Q 5/02; A61Q 5/12

(52) U.S. Cl. CPC A61K 8/0229; A61K 8/361; A61K 8/416; A61K 8/44; A61K 8/737; A61K 8/922; A61Q 5/02; A61Q 5/12

(57) ABSTRACT

A solid hair conditioner composition. The composition comprises the components (a) at least 2 wt % of a non-ionic or amphoteric surfactant; (b) at least 0.1 wt % of a hair conditioning agent; (c) at least 30 wt % of a carrier having a melting point of at least 45° C.; (d) at least 1 wt % of an emulsifier; and (e) at least 3 wt % water; wherein the composition is a solid throughout the range from 0° C. to 40° C. The composition may provide solid hair conditioner bars with good conditioning performance, feel and wear rate, a desired pH and which can be manufactured using conventional soap making methods and equipment. A method of method of manufacturing the solid hair conditioner composition and an emulsion for the preparation of the solid hair conditioner composition are also disclosed.

### FIELD

The present invention relates to a solid hair conditioner composition and a method of manufacturing the same. In particular the present invention relates to hair conditioner compositions which are particularly suitable for formulation into a solid bar form whilst retaining effectiveness and which can be produced using conventional solid soap bar-making equipment.

### BACKGROUND

In recent years, solid formulations of personal care products have become more popular with consumers due to the potential to reduce the environmental impact of such products. For example, solid personal care compositions can be supplied without the robust plastic packaging which liquid personal care compositions are typically supplied in. Such solid personal care compositions can be simply packaged in recyclable paper. Furthermore, solid personal care compositions may contain less water than equivalent liquid personal care compositions and therefore be lighter and require less energy to transport. Therefore such solid personal care compositions have the potential to drastically reduce the environmental impact of the packaging and transportation of personal care products.

These environmental benefits will only be obtainable if the solid personal care composition can perform its intended function as effectively as the equivalent liquid personal care composition and if it can be made in a sufficiently cost-effective manner, so that the product and its price to be attractive to consumers.

Soaps (that is, fatty acid salts) are an inexpensive solid personal cleansing composition which can be manufactured on a large scale efficiently. However, soap is not a product which is universally favoured by consumers. The pH of most soaps is about 9-10, whereas the natural pH of skin is about 5.5. Soap is an effective cleanser but is widely considered to be detrimental to skin. For this reason many consumers avoid soap and instead use “cleansing bars” which contain no soap or only a low proportion of soap.

Many cleansing bars (also called “detergent bars” or “syndet bars”) contain an isethionate surfactant as a major component. However, for reasons associated with the cost of ingredients, or for reasons associated with the manufacture, or both, cleansing bars are typically significantly more expensive than soap bars.

Previous attempts to manufacture solid hair shampoo and hair conditioner compositions which do not contain traditional soap (fatty acid salts) have met with limited success due to the difficultly in formulating effective solid hair shampoo and hair conditioner compositions which have an acceptable look and feel for the consumer and which can be manufactured in an efficient and cost-effective manner. This has been a particular problem in the production of solid hair conditioning compositions. Current solid hair conditioner bars have a high pH and/or can only be produced by relatively low throughput manufacturing processes, and have a much lower hair conditioning performance compared to common liquid hair conditioners.

### SUMMARY OF THE INVENTION

It is one aim of the present invention, amongst others, to provide a solid hair conditioner composition, a method of manufacturing a solid hair conditioner composition and an emulsion for the preparation of a solid hair conditioner composition that addresses at least one disadvantage of the prior art, whether identified here or elsewhere, or to provide an alternative to existing solid hair conditioner compositions, methods and emulsions. For instance, it may be an aim of the present invention to provide a solid hair conditioner composition which can be manufactured using conventional soap-making techniques and equipment.

According to aspects of the present invention, there is provided a solid hair conditioner composition, method and emulsion as set forth in the appended claims. Other features of the invention will be apparent from the dependent claims, and from the description which follows.

According to a first aspect of the present invention, there is provided a solid hair conditioner composition which comprises:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 3 wt % water;
   wherein the composition is a solid throughout the range from 0° C. to 40° C.

The solid hair conditioner composition of this first aspect is suitably produced by a method according to the second aspect of the present invention.

According to a second aspect of the present invention, there is provided a method of manufacturing a solid hair conditioner composition, the solid hair conditioner comprising:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 3 wt % water;
   wherein the composition is a solid throughout the range from 0° C. to 40° C.;
   and wherein the method comprises:
(i) preparing an emulsion containing the components (a) to (e), but with a greater mass of water present than in the solid hair conditioner composition, the temperature of the emulsion being at least 45° C.,
(ii) subjecting the emulsion to cooling and solidification, wherein the water content is reduced during the cooling and solidification to produce the solid hair conditioner composition having components (a) to (e) in the amounts defined.

The method of this second aspect suitably produces a solid conditioner composition according to the first aspect of the present invention.

The emulsion of step (i) is suitably an emulsion according to the third aspect of the present invention.

According to third aspect of the present invention, there is provided an emulsion for the preparation of a solid hair conditioner composition, the emulsion comprising:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 10 wt % water;
   wherein the emulsion is at a temperature of at least 45° C.

Suitably the emulsion of this third aspect is for producing a solid hair conditioner composition according to the first aspect of the present invention, suitably by a method according to the second aspect.

The method of the second aspect is suitably analogous to a conventional soap-making process which can be carried out on conventional soap-making equipment.

The inventors have found that the solid hair conditioner composition of the first aspect can be manufactured using such conventional soap-making processes and equipment and therefore may be more cost-effective to produce than known solid hair conditioner compositions. Furthermore, this efficiency of production may be achieved whilst obtaining an effective hair conditioner product with an advantageous feel, wear rate, pH and hair conditioning performance compared to known solid hair conditioner compositions.

The specific features described below apply to the first, second and third aspects of the present invention, where appropriate.

The solid hair conditioner composition comprises component (a) at least 2 wt % of a non-ionic or amphoteric surfactant. Component (a) may be provided by a mixture of non-ionic and amphoteric surfactants. Suitably component (a) is an amphoteric surfactant (or zwitterionic surfactants) or a mixture of amphoteric surfactants. Suitable amphoteric surfactants may be selected from alkyl betaines, alkyl amido betaines, alkyliminodipropionates, alkylamidopropyl hydroxy sultaines, alkylamphoacetates, alkylamphodiacetates, alkylamphopropionates, alkylamphodipropionates, alkyliminodipropionates and alkyliminodiacetates, or mixtures thereof. Suitably the amphoteric surfactant is a betaine. Suitably the amphoteric surfactant is an alkyl betaine or an alkyl amido betaine, suitably a C₈₋₁₆ alkyl betaine or a C₈₋₁₆ alkyl amido betaine, suitably a C₈₋₁₈ alkyl betaine, for example lauryl betaine.

The amphoteric surfactant may be selected from sodium lauroamphoacetate, disodium lauroamphoacetate, cocoamidopropyl betaine, cocoamidopropylhydroxy sultaine or lauryl betaine. Suitably component (a) is lauryl betaine.

Suitably component (a) is selected in each case to have a melting point of at least 45° C. Suitably component (a) has a melting point of at least 50° C., preferably of at least 55° C., more preferably of at least 60° C. In some preferred embodiments component (a) has a melting point of at least 65° C.

The non-ionic or amphoteric surfactant of component (a) provides at least 2 wt % of the solid hair conditioner composition, suitably at least 3 wt %, suitably at least 5 wt %, suitably at least 7 wt %, for example at least 9 wt % of the solid hair conditioner composition.

Suitably the non-ionic or amphoteric surfactant of component (a) provides up to 20 wt % of the solid hair conditioner composition, suitably up to 18 wt %, suitably up to 15 wt %, suitably up to 13 wt %, for example up to 12 wt % of the solid hair conditioner composition.

Suitably the non-ionic or amphoteric surfactant of component (a) provides form 2 to 20 wt % of the solid hair conditioner composition, suitably from 3 to 15 wt %, suitably from 7 to 15 w % or from 10 to 13 wt %, or from 6 to 10 wt % of the solid hair conditioner composition.

In some embodiments, wherein the solid hair conditioner composition is a conditioner without shampooing functionality, component (a) and component (b) are the only surfactants present in the composition. In such embodiments, the solid hair conditioner composition does not comprise an anionic surfactant.

The solid hair conditioner composition comprises component (b) at least 0.1 wt % of a hair conditioning agent. The hair conditioning agent may be selected from a silicone compound, a cationic polymer and a cationic surfactant or mixtures thereof.

A suitable silicone compound may be selected from diphenylsiloxy phenyl trimethicone, phenyl trimethicone, trimethylsiloxyphenyl dimethicone, phenyl propyl dimethyl siloxysilicate, diphenyl dimethicone and bisphenylpropydimethicone, dimethicone, dimethiconol and divinyldimethicone/dimethicone copolymer.

A suitable cationic polymer may be selected from guar hydroxypropyltrimonium chloride, hydroxypropyl guar hydroxypropyltrimonium chloride, polyquaternium-24, polyquaternium-10 and polyquaternium-67.

A suitable cationic surfactant conditioning agent may be quaternary ammonium compound, suitably selected from behentrimonium chloride, cetrimonium chloride and stearalkonium chloride.

Suitably component (b) is a cationic surfactant, suitably behentrimonium chloride.

The solid hair conditioner composition comprises component (b) at least 0.1 wt % of a hair conditioning agent, suitably at least 0.3 wt %, suitably at least 0.5 wt % of the hair conditioning agent.

Suitably the solid hair conditioner composition comprises up to 5 wt % of the hair conditioning agent, suitably up to 3 wt %, suitably up to 2 wt % of the hair conditioning agent, for example up to 1 wt %.

Suitably component (b) provides from 0.1 to 5 wt % of the solid hair conditioner composition, suitably from 0.5 to 3 wt %, suitably from 0.5 to 2 wt % or from 0.5 to 1 wt % of the solid hair conditioner composition, suitably wherein the hair conditioning agent is a cationic surfactant such as behentrimonium chloride.

The solid hair conditioner composition comprises component (c) at least 30 wt % of a carrier having a melting point of at least 45° C.

Suitably component (c) has a melting point of at least 50° C., preferably of at least 55° C., more preferably of at least 60° C. In some preferred embodiments component (c) has a melting point of at least 65° C.

Suitably the carrier, component (c), comprises one or more of a wax, a triglyceride, a fatty acid or a fatty alcohol. Suitably component (c) is selected from a triglyceride, a fatty acid, a fatty alcohol or a wax (including a mixture of any of these components). Suitably the carrier, component (c), is beeswax, or may be a triglyceride, fatty acid, fatty alcohol or a wax of vegetal origin.

Suitably component (c) is a triglyceride the fatty acid chains of which are residues of a C₈ fatty acid, or of higher fatty acids. Suitably the fatty acid chains of the triglyceride are residues of C₈-C₃₂ fatty acids, suitably of C₁₀-C₂₄ fatty acids, suitably of C₁₄-C₂₂ fatty acids and most preferably of C₁₆-C₂₀ fatty acids. Suitably the fatty acid chains of the triglyceride are residues of C₁₈ fatty acids.

Suitably component (c) is a saturated triglyceride. Suitably substantially all fatty acid chains in the triglyceride are saturated. Suitably component (c) is a hydrogenated triglyceride.

Suitably component (c) is a hydrogenated vegetable oil. Suitably component (c) comprises at least one of hydrogenated rapeseed oil, hydrogenated soybean oil, hydrogenated sunflower oil, hydrogenated palm oil, hydrogenated castor oil, hydrogenated safflower oil and hydrogenated peanut oil. Such oils may be employed singly or in admixture of two, three, four or more or all of these hydrogenated oils. Suitably these oils (summated if more than one is used) make up at least 50 wt %, preferably at least 60 wt %, preferably at least 70 wt %, preferably at least 80 wt %, preferably at least 90 wt %, and preferably 100 wt %, of the carrier, component (c).

Suitable carriers also include fatty acids, suitably C₈ fatty acid, or higher fatty acids; for example C₈-C₃₂ fatty acids, preferably C₁₀-C₂₄ fatty acids, preferably C₁₄-C₂₂ fatty acids and most preferably C₁₆-C₂₀ fatty acids. In some embodiments, the carrier is stearic acid.

Suitable carriers also include fatty alcohols, suitably C₁₆ fatty alcohol, or higher fatty alcohol; for example C₈-C₃₂ fatty alcohols, preferably C₁₀-C₂₄ fatty alcohols, preferably C₁₄-C₂₂ fatty alcohols and most preferably C₁₆-C₂₀ fatty alcohols.

The alkyl chain lengths of the fatty acid chains of the triglycerides, the fatty acids or the fatty alcohols discussed above (denoted Cx, for example C₁₈) is intended to represent the mean of the residues present in the triglycerides, the fatty acids or the fatty alcohols or to represent the chain length of more than half the weight of total fatty acid or fatty alcohol residues. If either of these definitions for a given alkyl chain length range is satisfied by a certain carrier component (c) then that carrier is included by the given alkyl chain length range.

Suitable waxes include waxes of vegetal origin, for example carnauba wax, and beeswax.

Suitably the solid hair conditioner composition comprises at least 35 wt % of the carrier, suitably at least 40 wt %, suitably at least 45 wt % or at least 50 wt % of the carrier.

Suitably the solid hair conditioner composition comprises up to 80 wt % of the carrier, suitably up to 75 wt %, suitably up to 70 wt % or up to 65 wt % of the carrier.

Suitably component (c) provides from 30 to 80 wt % of the solid hair conditioner composition, suitably from 30 to 70 wt %, suitably from 40 to 70 wt %, suitably from 50 to 65 wt % of the solid hair conditioner composition, suitably wherein the carrier is a hydrogenated vegetable oil or wherein the carrier is stearic acid.

Suitably the wt % of the carrier component (c) exceeds the wt % of the non-ionic or amphoteric surfactant component (a) in the solid hair conditioning composition.

The amount and type of the carrier component (c) may be adjusted within the ranges and options described above to achieve a desired hardness, wear rate and appearance of the solid hair conditioner composition.

The solid hair conditioner composition comprises component (d) at least 1 wt % of an emulsifier.

Suitably the emulsifier, component (d), has an HLB value of at least 2, preferably at least 3.

Suitably the emulsifier component (d) has an HLB value of up to 6, suitably up to 5.

Suitably the emulsifier component (d) has an HLB of from 2 to 6, suitably from 3 to 6, for example an HLB value of from 3 to 5 or from 3.2 to 4.2.

The desired HLB value may be that of a single emulsifier or may be achieved by selection of two or more emulsifiers which in combination—calculated as a weighted average taking into account their individual HLB values and their amounts in the blend—achieve the desired HLB value, within the preferred range 2 to 6, 3 to 6 or 3 to 5. Such a weighted average for emulsifiers A1, A2, A3 . . . can be calculated using the equation:

$$( mass of A 1 \times HLB of A 1 ) + ( mass of A 2 \times HLB of A 2 ) + ( mass of A 3 \times HLB of A 3 ) + … mass of A 1 + mass of A 2 + mass of A 3 + …$$

Suitable emulsifiers (d) include sorbitan fatty acid esters and ethoxylated sorbitan fatty acid esters. Suitable sorbitan fatty acid esters, non-ethoxylated, include sorbitan monolaurate, sorbitan monooleate, sorbitan monopalmitate, sorbitan monostearate and sorbitan tristearate. Suitable ethoxylated sorbitan fatty acid esters include polyoxyethylene sorbitan monolaurate, polyoxyethylenesorbitan monooleate, polyoxyethylene sorbitan monopalmitate, polyoxyethylene sorbitan monostearate and polyoxyethylene sorbitan tristearate.

Suitable emulsifiers (d) include glycerol esters of fatty acids; that is, esters of fatty acids and glycerol or polyglycerol. A very wide range of suitable glycerol esters may be made by reaction of glycerol or polyglycerol and C₆-C₃₆ fatty acids, for example C₁₂-C₂₄ fatty acids. The fatty acids may be supplied to the esterification reaction as fatty acids or as precursor ester compounds, for example triglycerides. Examples suitable for use in this invention include glycerol monostearate, glycerol monooleate, glycerol palmitate and glycerol monolaurate.

Component (d) may be selected from sorbitan fatty acid esters, ethoxylated sorbitan fatty acid esters and glycerol or polyglycerol esters of fatty acids.

In some embodiments, the emulsifier component (d) is glycerol monooleate (which may be referred to as glyceryl monooleate or glycerol oleate). Glycerol monooleate has a HLB value of around 4.

In some embodiments, the emulsifier component (d) is glycerol monostearate (which may be referred to as glyceryl monostearate or glycerol stearate). Glycerol monostearate has a HLB value of around 4.

In some embodiments, the emulsifier component (d) is polyglycerol-4 oleate. Polyglycerol-4 oleate has an HLB value of around 5.

The inventors have found that using an emulsifier component (d) having an HLB value in the ranges described above, for example glycerol monooleate having an HLB of around 4 or a polyglycerol-4 oleate having an HLB of around 5, may be particularly advantageous in emulsifying the components of the solid hair conditioner composition effectively to form the emulsion of the third aspect which in turn allows the formation of the solid hair conditioner composition having the desired feel, wear rate and conditioning performance using a method of the second aspect.

Suitably the emulsifier component (d) provides at least 2 wt % of the solid hair conditioner composition, suitably at least 3 wt %, suitably at least 4 wt % or at least 5 wt % of the solid hair conditioner composition.

Suitably the emulsifier component (d) provides up to 12 wt % of the solid hair conditioner composition, suitably up to 10 wt %, suitably up to 9 wt % or up to 8 wt % of the solid hair conditioner composition.

Suitably the emulsifier component (d) provides from 2 to 12 wt % of the solid hair conditioner composition, suitably from 4 to 10 wt %, suitably from 4 to 9 wt % or from 5 to 8 wt % of the solid hair conditioner composition.

The solid hair conditioner composition comprises component (e) at least 3 wt % water. Suitably component (e) provides at least 6 wt % of the solid hair conditioner composition, suitably at least 8 wt % or at least 10 wt % of the solid hair conditioner composition.

Suitably the water component (e) provides up to 20 wt % of the solid hair conditioner composition, suitably up to 18 wt %, suitably up to 16 wt % or up to 14 wt % of the solid hair conditioner composition.

Suitably the water component (e) provides from 5 to 20 wt % of the solid hair conditioner composition, suitably from 5 to 18 wt %, suitably from 8 to 16 wt % or from 10 to 14 wt % of the solid hair conditioner composition.

The content of water (e) in the solid hair conditioner composition may be a result of the method of manufacture of the composition. The solid hair conditioner composition is suitably manufactured by a conventional soap-making process which involves forming an emulsion containing excess water, compared with the water content of the solid hair conditioner composition, and then spray drying the emulsion to form the solid composition comprising the amounts of water discussed above. Having the stated amounts of water in the solid hair conditioner composition advantageously allows the composition to be formed by such conventional soap-making processes using the same equipment.

Suitably components (a) to (e) provide at least 70 wt %, at least 80 wt % or at least 90 wt % of the solid hair conditioner composition. Components (a) to (e) may provide up to 100 wt % of the solid hair conditioner composition.

In some embodiments, the solid hair conditioner composition comprises:
(a) from 3 to 15 wt % of a non-ionic or amphoteric surfactant;
(b) 0.5 to 3 wt % of a hair conditioning agent;
(c) 30 to 70 wt % of a carrier having a melting point of at least 45° C.;
(d) from 4 to 10 wt % of an emulsifier; and
(e) from 5 to 18 wt % water;
   wherein the composition is a solid throughout the range from 0° C. to 40° C.

In some embodiments, the solid hair conditioner composition comprises:
(a) from 3 to 15 wt % of an amphoteric surfactant;
(b) 0.5 to 3 wt % of a cationic surfactant hair conditioning agent;
(c) 30 to 70 wt % of a hydrogenated vegetable oil carrier having a melting point of at least 45° C.;
(d) from 4 to 10 wt % of an emulsifier having an HLB of from 3 to 6; and
(e) from 5 to 18 wt % water;
   wherein the composition is a solid throughout the range from 0° C. to 40° C.

In some embodiments, the solid hair conditioner composition comprises:
(a) from 3 to 15 wt % of an amphoteric surfactant;
(b) 0.5 to 3 wt % of a cationic surfactant hair conditioning agent;
(c) 30 to 70 wt % of stearic acid as a carrier;
(d) from 4 to 10 wt % of an emulsifier having an HLB of from 3 to 6; and
(e) from 5 to 18 wt % water;
   wherein the composition is a solid throughout the range from 0° C. to 40° C.

### Solid Combination Hair Shampoo and Conditioner Composition

In some embodiments, the solid hair conditioner composition includes an additional surfactant and has a hair cleaning functionality as well as a hair conditioning functionality. Therefore such embodiments may provide a solid combination hair shampoo and hair conditioner composition (which may be referred to as a solid 2-in-1 composition or a solid hair conditioning shampoo composition).

In such embodiments, the surfactant is suitably a non-soap anionic surfactant and therefore the solid combination hair shampoo and hair conditioner composition comprises a non-soap anionic surfactant component (f.

Suitable non-soap anionic surfactants may be selected from a sulfate, sulfonate, amphoacetate, sulfoacetate, sulfosuccinate, phosphate or carboxylate non-soap anionic surfactant; suitably selected in each case to have a melting point of at least 45° C.

Suitably component (f) may have a melting point of at least 50° C., preferably of at least 55° C., more preferably of at least 60° C. In some preferred embodiments component (f) has a melting point of at least 65° C.

Sulfate non-soap anionic surfactants may include ammonium lauryl sulfate, sodium lauryl sulfate (SLS), sodium coco sulfate (SCS), fatty alcohol sulfates, and alkyl-ether sulfates, for example sodium laureth sulfate (SLES), sodium myreth sulfate and polyoxyethylene fatty alcohol ether sulfates.

Sulfonate non-soap anionic surfactants may include alkyl sulfonates, alkylbenzene sulfonates, alkenyl sulfonates, alkyl succinate sulfonates, alkylphenol sulfonates perflouoralkylsulfonates and acyl isethionates. A suitable alkyl succinate sulfonate is disodium laureth sulfosuccinate.

Amphoacetates, sulfoacetates, and sulfosuccinates non-soap anionic surfactants may include sodium lauryl sulfoacetate and disodium laureth sulfosuccinate.

Phosphate non-soap anionic surfactants may include alkyl-aryl ether phosphates and alkyl ether phosphates.

Carboxylate non-soap anionic surfactants may include sarcosinates, for example sodium lauroyl sarcosinate, and carboxylate-based fluorosurfactants, for example perfluorononanoate and perfluorooctanoate (PFOA or PFO). Suitable carboxylate non-soap anionic surfactants may include acyl glutamates, for example sodium cocoyl glutamate or sodium stearoyl glutamate.

Preferred non-soap anionic surfactants (f) for use on this invention are acyl isethionates.

An acyl isethionate for use in this invention is suitably of formula (I):

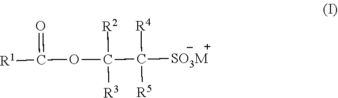

wherein R¹ is an alkyl or alkenyl group having from 8 to 24 carbon atoms;
R² is a hydrogen atom or an alkyl or alkenyl group having from 1 to 8 carbon atoms;
R³ is a hydrogen atom or an alkyl or alkenyl group having from 1 to 8 carbon atoms;
R⁴ is a hydrogen atom or an alkyl or alkenyl group having from 1 to 8 carbon atoms;
R⁵ is a hydrogen atom or an alkyl or alkenyl group having from 1 to 8 carbon atoms;
and M⁺ is a cation.

Preferably M⁺ represents an optionally substituted ammonium cation or, most preferably, a metal cation. Suitable ammonium cations include NH₄⁺ and the ammonium cation of triethanolamine.

Suitable metal cations include alkali metal cations, for example sodium, lithium and potassium cations, and alkaline earth metal cations, for example calcium and magnesium cations (it should be noted here that when M⁺ represents an alkaline earth metal cation M⁺ has a double positive charge and the compound has two anions). Preferably M⁺ represents a potassium cation, or, especially, a sodium cation.

R¹ may be an alkyl group or an alkenyl group. Preferably R¹ is an alkyl group. In some embodiments component (f) may comprise a surfactant derived from a mixture of fatty acids to form a mixture of compounds of formula (I) in which R¹ may be different.

R¹ is preferably the residue of a fatty acid. Fatty acids obtained from natural oils often include mixtures of fatty acids. For example the fatty acid obtained from coconut oil contains a mixture of fatty acids including C₁₂ lauric acid, C₁₄ myristic acid, C₁₆ palmitic acid, C₈ caprylic acid, and C₁₈ stearic and oleic.

R¹ may include the residue of one or more naturally occurring fatty acids and/or of one or more synthetic fatty acids. In some preferred embodiments R¹ consists essentially of the residue of a single fatty acid.

Examples of carboxylic acids from which R¹ may be derived include coco acid, butyric acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic lignoceric acid, naturally occurring fatty acids such as those obtained from coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Most preferably R¹ comprises the residue of lauric acid, that is a saturated fatty acid having 12 carbon atoms or the residue of mixed fatty acids derived from coconut oil, in which lauric acid chains predominate.

R² is preferably a hydrogen atom or an alkyl or alkenyl group having from 1 to 4 carbon atoms. Most preferably R² is a hydrogen atom.

R³ is preferably a hydrogen atom or an alkyl or alkenyl group having from 1 to 4 carbon atoms. Most preferably R³ is a hydrogen atom.

R⁴ is preferably a hydrogen atom or an alkyl or alkenyl group having from 1 to 4 carbon atoms. Most preferably R² is a hydrogen atom.

R⁶ is preferably a hydrogen atom or an alkyl or alkenyl group having from 1 to 4 carbon atoms. Most preferably R² is a hydrogen atom.

In some embodiments three of the entities R², R³, R⁴ and R⁵ are hydrogen and only one is an alkyl or alkenyl group having from 1 to 8 carbon atoms. In such embodiments preferably R³, R⁴ and R⁵ are hydrogen and R² is the alkyl or alkenyl group.

However in especially preferred embodiments R², R³, R⁴ and R⁵ are all hydrogen.

In some especially favoured embodiments component (f) comprises one or more or all of sodium lauroyl isethionate (SLI), sodium oleoyl isethionate and sodium cocoyl isethionate (SCI), which is closely related to but not identical to SLI.

Most preferably the component (f) of the composition of the present invention comprises or consists of sodium cocoyl isethionate and/or sodium lauroyl isethionate.

An acyl isethionate may serve as the only constituent of component (f). However, in some embodiments component (f) may comprise an acyl isethionate in admixture with one or more further non-soap anionic surfactants, for example any of those defined above. In such admixture embodiments acyl isethionate(s) suitably constitute(s) at least 50 wt % of, and preferably at least 65 wt %, of component (f).

Suitably component (f) provides at least 10 wt % of the solid combination hair shampoo and hair conditioner composition, suitably at least 12 wt %, suitably at least 13 wt % or at least 15 wt % of the composition.

Suitably component (f) provides up to 40 wt % of the solid combination hair shampoo and hair conditioner composition, suitably up to 30 wt %, suitably up to 25 wt % or up to 22 wt % of the composition.

Suitably component (f) provides from 10 to 40 wt % of the solid combination hair shampoo and hair conditioner composition, suitably from 12 to 30 wt % or from 15 to 25 wt % of the composition.

In such embodiments, the non-ionic or amphoteric surfactant of component (a) suitably provides form 2 to 10 wt % of the solid combination hair shampoo and hair conditioner composition, suitably from 2 to 5 wt %, suitably from 2 to 4 w % of the composition.

In such embodiments, the solid combination hair shampoo and hair conditioner composition suitably comprises component (b) in the amounts described above.

In such embodiments, component (c) suitably provides from 30 to 60 wt % of the solid combination hair shampoo and hair conditioner composition, suitably from 30 to 50 wt %, suitably from 35 to 45 wt %, of the composition, suitably wherein the carrier is a hydrogenated vegetable oil or stearic acid.

In such embodiments, the solid combination hair shampoo and hair conditioner composition suitably comprises component (d) in the amounts described above.

In such embodiments, the emulsifier, component (d), may have an HLB in the ranges described above.

Alternatively, it may be beneficial for the solid combination hair shampoo and hair conditioner composition to include an emulsifier with a higher HLB. Therefore, in such embodiments, component (d) suitably has an HLB in the range of from 7 to 11, suitably from 8 to 10, for example an HLB value of around 9.

In such embodiments, the solid combination hair shampoo and hair conditioner composition suitably comprises component (e) in the amounts described above.

In such embodiments, the solid combination hair shampoo and hair conditioner composition comprises:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier;
(e) at least 3 wt % water; and
(f) at least 10 wt % of a non-soap anionic surfactant;
   wherein the composition is a solid throughout the range from 0° C. to 40° C.

In such embodiments, the solid combination hair shampoo and hair conditioner composition comprises:
(a) from 2 to 10 wt % of a non-ionic or amphoteric surfactant;
(b) 0.5 to 3 wt % of a hair conditioning agent;
(c) 30 to 50 wt % of a carrier having a melting point of at least 45° C.;
(d) from 4 to 10 wt % of an emulsifier; and
(e) from 5 to 18 wt % water; and
(f) from 12 to 30 wt % of a non-soap anionic surfactant;
   wherein the composition is a solid throughout the range from 0° C. to 40° C.

In such embodiments, the solid combination hair shampoo and hair conditioner composition comprises:
(a) from 2 to 10 wt % of an amphoteric surfactant;
(b) 0.5 to 3 wt % of a cationic surfactant hair conditioning agent;
(c) 30 to 50 wt % of a hydrogenated vegetable oil carrier having a melting point of at least 45° C.;
(d) from 4 to 10 wt % of an emulsifier; and
(e) from 5 to 18 wt % water; and
(f) from 12 to 30 wt % of an acyl isethionate surfactant;
   wherein the composition is a solid throughout the range from 0° C. to 40° C.

In such embodiments, the solid combination hair shampoo and hair conditioner composition comprises:
(a) from 2 to 10 wt % of an amphoteric surfactant;
(b) 0.5 to 3 wt % of a cationic surfactant hair conditioning agent;
(c) 30 to 50 wt % of stearic acid as a carrier;
(d) from 4 to 10 wt % of an emulsifier; and
(e) from 5 to 18 wt % water; and
(f) from 12 to 30 wt % of an acyl isethionate surfactant;
   wherein the composition is a solid throughout the range from 0° C. to 40° C.

The solid hair conditioner composition of the present invention may be in the form of noodles, pellets, flakes or powder. Such forms of the solid hair conditioner composition may be suitable for further processing with further ingredients into a solid hair conditioner product, such as block, bar or tablet.

The solid hair conditioner composition of the present invention may be in the form of a block, bar or tablet.

Suitably, the solid hair conditioner composition is soap-free, i.e. does not contain any fatty acid soap (salts of a fatty acid).

Suitably, the solid hair conditioner composition does not contain any starch.

Suitable, the solid hair conditioner composition does not contain any soap or starch.

### Further Components

Optional further components may include (without limitation) polyhydric alcohols, chelants and fatty alcohols. Further components may offer manufacturing benefits, for example foam control during manufacture, and/or end product user benefits, for example foam control during washing, or emollience. Further components may preferably be present in total in an amount of up to 30 wt % of the solid hair conditioner composition.

Suitable polyhydric alcohols include C₂-C₄ alcohols, especially glycols and glycerols, for example monopropylene glycol and glycerine. Polyhydric alcohols, when present, may suitably be present in total in an amount of up to 15 wt % of the solid hair conditioner composition, preferably up to 10 wt % or up to 5 wt %.

Suitable chelants include DTPA (diethylenetriaminepentaacetic acid), HEDP (hydroxyethylidene diphosphonic acid), NTA (nitrilotriacetic acid), EDTA (ethylenediaminetetraacetic acid), EDDS (ethylenediamine-N,N′-disuccinic acid), GLDA (N,N-dicarboxymethyl glutamic acid tetrasodium salt) (tetrasodium glutamate diacetate), and PDTA (propylenedinitrilotetraacetic acid); and chelants which are analogues of any of the foregoing. Chelants, when present, may suitably be present in total in an amount of up to 0.5 wt % of the solid hair conditioner composition, preferably up to 0.3 wt %.

It will be understood by the skilled reader that components of the compositions of the invention—notably components (a) and (b)—may be derived from natural sources and may contain a distribution of molecules about a mean carbon value. Such components are expected to melt over a temperature range, rather than have a sharp melting point. The melting point definitions defined in this specification refer to the temperatures at which a material is completely melted. The method described in the paper Determination of melting point of vegetable oils and fats by differential scanning calorimetry (DSC) technique by Renata Tieko Nassu and Lireny Aparecida Guaraldo Gonçalves, in the Grasas y Aceites Vol. 50. Fase. 1 (1999), 16-22 may be consulted. DSC as described in this paper can be used to determine when a material is completely in the liquid state.

### Method of Manufacturing the Solid Hair Conditioner Composition

The second aspect of the present invention provides a method of manufacturing a solid hair conditioner composition, the solid hair conditioner composition comprising:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 3 wt % water;
   wherein the composition is a solid throughout the range from 0° C. to 40° C.;
   and wherein the method comprises:
(i) preparing an emulsion containing the components (a) to (e), but with a greater mass of water present than in the solid hair conditioner composition, the temperature of the emulsion being at least 45° C.,
(ii) subjecting the emulsion to cooling and solidification, wherein the water content is reduced during the cooling and solidification to produce the solid hair conditioner composition having components (a) to (e) in the amounts defined.

The solid hair conditioner composition produced by this method may have any of the suitable features and advantages described above.

Suitably components (a) to (e) make up at least 70 wt %, suitably at least 80 wt %, at least 90 wt % or at least 95 wt % of the emulsion. Components (a) to (e) may provide up to 100 wt % of the weight of the emulsion.

Suitably the steps of the method of this second aspect are carried out in the order of step (i) followed by step (ii).

Suitably, the emulsion in step (i) is at a temperature at which it is a free-flowing liquid. Suitably the emulsion at a temperature of at least 45° C. is a free-flowing liquid.

Suitably component (a) has a melting point of at least 50° C., the carrier (b) has a melting point of at least 50° C., and the emulsion is at a temperature of at least 50° C., at which temperature the emulsion is a free-flowing liquid.

Suitably component (a) has a melting point of at least 55° C., the carrier (b) has a melting point of at least 55° C., and the emulsion is at a temperature of at least 55° C., at which temperature the emulsion is a free-flowing liquid.

Suitably component (a) has a melting point of at least 60° C., the carrier (b) has a melting point of at least 60° C., and the emulsion is at a temperature of at least 60° C., at which temperature the emulsion is a free-flowing liquid.

Suitably in step (i) of the method, the emulsion is at a temperature of at least 60° C., suitably at least 70° C., suitably at least 75° C., suitably at least 80° C., suitably at least 85° C.

Suitably in step (i) of the method, the emulsion is at a temperature of up to 100° C., suitably up to 95° C., suitably up to 90° C.

Suitably in step (i) of the method, the emulsion is at a temperature in the range 80° C. to 95° C., suitably in the range 85° C. to 90° C.

Suitably the emulsion is an oil-in-water emulsion.

The emulsion may be prepared according to the methods disclosed in WO2014170641.

Suitably component (a) is added to water (e) which is at a selected elevated temperature. The emulsifier (c) is suitably added at the same time as the surfactant (a) or subsequently. The carrier (b) suitably is added to the water after the addition of the surfactant (a) and emulsifier (c). The addition to the water of the emulsifier before the carrier means that the emulsifier is present to immediately start to emulsify the carrier and prevent it from forming a film which otherwise could disrupt the process.

Other ingredients, if present, are preferably added whenever convenient. Frequently it is suitable to add them to the water with the surfactant, or before.

Suitably the emulsion is thoroughly stirred in step (i) to effectively emulsify and mix the components (a) to (e).

It is important in the method of this second aspect that the emulsion contains excess water, compared with the water content of the solid hair conditioner composition. The presence of excess water allows the temperature of the mass to be controlled. Water is typically lost from the emulsion by evaporation during the cooling and solidification. This may be carried out by using a spray dryer or any other method in which the emulsion is separated into droplets or pellets which are exposed to air and may rapidly lose heat, as they cool.

Suitably the water component (e) provides at least 12 wt % of the emulsion, suitably at least 16 wt % or at least 20 wt % of the emulsion.

Suitably the water component (e) provides up to 40 wt % of the emulsion, suitably up to 30 wt %, suitably up to 26 wt % or up to 24 wt % of the emulsion.

Suitably the water component (e) provides from 12 to 30 wt % of the emulsion, suitably from 16 to 26 wt %, suitably from 18 to 26 wt % or from 20 to 24 wt % of the emulsion.

Preferably the mass of water used for the process of manufacture is at least 30% higher than the mass of water present in solid hair conditioner composition; preferably at least 40% higher; preferably at least 50% higher; preferably at least 60% higher, and in some preferred embodiments at least 70% higher. In some especially preferred embodiments the mass of water is at least 100% higher than the mass of water in the solid hair conditioner composition, and may be at least 125% higher.

Suitably the mass of water initially present in the emulsion in step (i) is at least 40% higher than the mass of water present in the solid hair conditioner composition after step (ii).

Preferably the mass of water used for the process of manufacture is not more than 300% higher than the mass of water present in the solid hair conditioner composition; preferably not more than 250% higher; preferably not more than 200% higher or more than 100% higher.

It should be noted that the above definitions compare the mass of water used in the process of manufacture—that is, the mass of water in the emulsion—with the mass of water present in the solid hair conditioner composition. They do not define the mass of water in the emulsion expressed in terms of total mass of components in the emulsion, nor the mass of water in the solid hair conditioner composition expressed in terms of total mass of components in the solid hair conditioner composition.

In step (ii) of the method of this second aspect, the emulsion is cooled and solidified to form particulates, for example noodles, pellets, flakes or powder or the like, which may then be consolidated into a mass and separated into blocks, bars, tablets or the like.

Therefore the method of this second aspect may comprise a step (iii) of consolidating the solid hair conditioner composition into a block, bar or tablet of the solid hair conditioner composition. Step (iii) may be carried out by plodding on appropriate equipment. Methods and equipment for consolidating the solid hair conditioner composition into a block, bar or tablet, for example by plodding, are known in the art. Plodding may produce a billet of the solid hair conditioner composition. Step (iii) may involve stamping such a billet into the blocks, bars or tablets of the solid hair conditioner composition, suitably using chilled dies.

Ultimately the product offered to consumers of conditioner bars may be a bar or tablet of conventional size, for example 50-200 g; typically 80-130 g. Alternatively the product could be provided in the form of noodles, pellets, particles, flakes, chips or the like from which a manufacturer may make such bars or tablets.

The particulates and the resulting solid consolidated mass are suitably homogeneous in their microstructure, not heterogeneous. In preferred embodiments there is no post-solidification addition of any components to the solid hair conditioner material; the entire process preferably occurs in the liquid phase.

The manufacture of solid non-soap personal care bars by methods used up to this time has been slow, compared to the manufacture of soap bars. This is one reason why syndet bars are expensive, in comparison with soap bars. The manufacture of solid hair conditioner bars also suffers from this drawback. This high cost of manufacturing further solid hair conditioner bars exacerbated by the relatively high cost of the ingredients, especially when said bars wear too rapidly, wasting such relatively expensive ingredients, or wear too slowly to provide sufficient active ingredients to the hair in use. By virtue of the ingredients and process parameters used the method of the second aspect is essentially able to use the equipment and techniques of conventional soap-making, leading to significantly higher production rates for the manufacture of solid hair conditioner bars than would be expected. Furthermore, the solid hair conditioner bars of the present invention may advantageously have an optimal pH to minimise skin irritancy and also an appropriate wear rate to provide sufficient release of active ingredients for the desired conditioning performance during use whilst minimising wastage of said active ingredients.

The invention will now be further described by way of illustration with reference to the following examples.

### EXAMPLES

### Example 1

A solid hair conditioner bar Example 1 was formed using the following ingredients and the following procedure. The wt % of ingredients are given in the table below in the amounts used to form an emulsion (second column) and the amounts present in the solid hair conditioner bar after formation (third column), which involved removing some of water during the spray drying step.

| | wt % as added | wt % in |
|---|---|---|
| Ingredient | (in emulsion) | product |
| Hydrogenated vegetable oil | 54.36 | 60.96 |
| Water | 21.53 | 12.00 |
| Lauryl betaine | 10.20 | 11.44 |
| Glyceryl oleate MB | 6.88 | 7.72 |
| Glycerine | 3.56 | 3.99 |
| Sodium chloride | 2.38 | 2.67 |
| Behentrimonium chloride | 0.80 | 0.90 |
| Isopropyl alcohol | 0.20 | 0.22 |
| Tetrasodium glutamate diacetate | 0.09 | 0.11 |
| Total | 100.00 | 100.00 |

Lauryl Betaine was provided by using 34 wt % Mackam LAB 30 which contains 30 wt % lauryl betaine, 63 wt % water and 7 wt % salt. The majority of the water in the table above was provided by the water present in the Mackam LAB 30. The remainder of the water was provided by water present in the source of tetrasodium Glutamate Diacetate. The behentrimonium chloride was provided in 20% IPA. Therefore the isopropyl alcohol listed above was provided by the source of behentrimonium chloride.

The hydrogenated vegetable oil used was VGB 506 MOLTEN obtained from ADM-SIO.

The glyceryl oleate MB used was Primesurf GMO RSPO MB obtained from Prime Surfactants. The HLB of the glycerol oleate MB is 3.8.

The behentrimonium chloride used was Genamin KDMP supplied by Clariant or Primesurf BTAC80 supplied by Prime Surfactants.

The lauryl betaine (Mackam LAB 30) comprising the water was combined with the glyceryl oleate and heated to 90° C. The hydrogenated vegetable oil was added to this mixture with the other ingredients and the mixture was worked for about 5 hours, whilst still molten. The resulting emulsion, a readily flowable liquid, was then pumped twice through a needle plate having a 0.5 mm mesh and allowed to solidify as particles. During this phase of the processing, water was lost by evaporation and the water content was thereby reduced to approximately 12 wt % based on the resulting solid hair conditioner composition. The particles were allowed to cool before being passed to a proprietary plodder. A typical plodder is the Duplex Model M400-2/M400-4 plodder manufactured by Mazzoni S.P.A. of Busto Arsizio, Italy. During plodding the temperature of the solid hair conditioner composition was maintained between 38° C. and 42° C. The billet produced by plodding, still at 38-42° C., was stamped into bars on chilled dies. The solid hair conditioner composition produced using this apparatus had good processability on this apparatus, which is indicative of being suitable for high throughput manufacture using conventional soap making equipment on a larger scale.

The pH of the solid hair conditioner composition was in the range 7-8, as tested at 10 wt % solution at 40° C. of the composition in water using a commercial pH meter.

The resulting products were of high quality, perfectly suitable for use. The solid hair conditioner composition bar had an acceptable wear rate for effective conditioner performance whilst having sufficient longevity on repeated usage.

### Example 2

The solid hair conditioner bar of Example 2 was formed from the following ingredients and using the same procedure described above for Example 1.

| | wt % as added | wt % in |
|---|---|---|
| Ingredient | (in emulsion) | product |
| Hydrogenated vegetable oil | 54.36 | 60.96 |
| Water | 21.53 | 12.00 |
| Lauryl betaine | 10.20 | 11.44 |
| Glyceryl mono stearate | 6.88 | 7.72 |
| Glycerine | 3.56 | 3.99 |
| Sodium chloride | 2.38 | 2.67 |
| Behentrimonium chloride | 0.80 | 0.90 |
| Isopropyl alcohol | 0.20 | 0.22 |
| Tetrasodium glutamate diacetate | 0.09 | 0.11 |
| Total | 100.00 | 100.00 |

The glyceryl monostearate used was Primesurf GMS NE40 Granules RSPO MB supplied by Prime Surfactants Ltd. The HLB of the glyceryl monostearate is approximately 3.8.

As with Example 1, the resulting solid hair conditioner composition bars were of high quality, perfectly suitable for use, had an acceptable wear rate for effective conditioner performance whilst having sufficient longevity on repeated usage, has good processability and had a similar pH to Example 1.

### Example 3

The solid hair conditioner bar of Example 3 was formed from the following ingredients and using the same procedure described above for Example 1.

| | wt % as added | wt % in |
|---|---|---|
| Ingredient | (in emulsion) | product |
| Hydrogenated vegetable oil | 54.36 | 60.96 |
| Water | 21.53 | 12.00 |
| Lauryl betaine | 10.20 | 11.44 |
| Glyceryl mono stearate | 3.44 | 3.86 |
| Glyceryl oleate MB | 3.44 | 3.86 |
| Glycerine | 3.56 | 3.99 |
| Sodium chloride | 2.38 | 2.67 |
| Behentrimonium chloride | 0.80 | 0.90 |
| Isopropyl alcohol | 0.20 | 0.22 |
| Tetrasodium glutamate diacetate | 0.09 | 0.11 |
| Total | 100.00 | 100.00 |

As with Example 1, the resulting solid hair conditioner composition bars were of high quality, perfectly suitable for use, had an acceptable wear rate for effective conditioner performance whilst having sufficient longevity on repeated usage, has good processability and had a similar pH to Example 1.

### Example 4

The solid hair conditioner bar of Example 4 was formed from the following ingredients and using the same procedure described above for Example 1.

| | wt % as added | wt % in |
|---|---|---|
| Ingredient | (in emulsion) | product |
| Hydrogenated vegetable oil | 54.36 | 60.95 |
| Water | 21.52 | 12.00 |
| Lauryl betaine | 10.2 | 11.44 |
| Durosoft PG4L | 6.88 | 7.72 |
| Glycerine | 3.56 | 3.99 |
| Sodium chloride | 2.38 | 2.67 |
| Behentrimonium chloride | 0.80 | 0.90 |
| Isopropyl alcohol | 0.20 | 0.22 |
| Tetrasodium glutamate diacetate | 0.10 | 0.11 |
| Total | 100.00 | 100.00 |

Durosoft PK4L-SG is a Trade Mark of Stephenson Group Limited of Leeds, UK. It is an ester of palm kernel oil and polyglycerol-4 (average Mw=250) and has an HLB value of between 8-10.

As with Example 1, the resulting solid hair conditioner composition bars were of high quality, perfectly suitable for use, had an acceptable wear rate for effective conditioner performance whilst having sufficient longevity on repeated usage, has good processability and had a similar pH to Example 1. This solid hair conditioner bar of Example 4 had the desired firmness and very low amounts of grit.

### Example 5

The solid hair conditioner bar of Example 5 was formed from the following ingredients and using the same procedure described above for Example 1.

| | wt % as added | wt % in |
|---|---|---|
| Ingredient | (in emulsion) | product |
| Hydrogenated vegetable oil | 54.36 | 60.94 |
| Water | 21.49 | 12.00 |
| Citric acid | 0.04 | 0.04 |
| Lauryl betaine | 10.20 | 11.43 |
| Glyceryl oleate MB | 6.88 | 7.71 |
| Glycerine | 3.56 | 3.99 |
| Sodium chloride | 2.38 | 2.67 |
| Behentrimonium chloride | 0.80 | 0.90 |
| Isopropyl alcohol | 0.20 | 0.22 |
| Tetrasodium glutamate diacetate | 0.09 | 0.10 |
| Total | 100.00 | 100.00 |

The citric acid was provided by using 0.08 wt % of a 50 wt % solution of citric acid in water. The citric acid was used to adjust the pH of the composition.

As with Example 1, the resulting solid hair conditioner composition bars were of high quality, perfectly suitable for use, had an acceptable wear rate for effective conditioner performance whilst having sufficient longevity on repeated usage and has good processability. The pH of the bar of Example 5 was approximately 6.

### Example 6

The solid hair conditioner bar of Example 6 was formed from the following ingredients and using the same procedure described above for Example 1. 39 wt % of Mackam LAB 30 was used to provide the 11.70 wt % of lauryl betaine, 24.57 wt % water and 2.73 wt % sodium chloride.

| | wt % as added | wt % in |
|---|---|---|
| Ingredient | (in emulsion) | product |
| Hydrogenated vegetable oil | 49.36 | 57.66 |
| Water | 24.67 | 12.00 |
| Lauryl betaine | 11.70 | 13.67 |
| Glyceryl oleate MB | 6.88 | 8.04 |
| Glycerine | 3.56 | 4.16 |
| Sodium chloride | 2.73 | 3.19 |
| Behentrimonium chloride | 0.8 | 0.94 |
| Isopropyl alcohol | 0.20 | 0.23 |
| Tetrasodium glutamate diacetate | 0.1 | 0.12 |
| Total | 100.00 | 100.00 |

As with Example 1, the resulting solid hair conditioner composition bars were of high quality, perfectly suitable for use, had an acceptable wear rate for effective conditioner performance whilst having sufficient longevity on repeated usage, although bars were slightly softer and their wear rate was higher than the bars of Example 1.

The solid hair conditioner composition bars of Example 6 had good processability and had a similar pH to Example 1.

### Example 7

The solid hair conditioner bar of Example 7 was formed from the following ingredients and using the same procedure described above for Example 1. 37.5 wt % of Mackam LAB 30 was used to provide the 11.25 wt % of auryl betaine, 23.63 wt % water and 2.63 wt % sodium chloride.

| | wt % as added | wt % in |
|---|---|---|
| Ingredient | (in emulsion) | product |
| Hydrogenated vegetable oil | 49.36 | 56.95 |
| Water | 23.73 | 12.00 |
| Lauryl betaine | 11.25 | 12.98 |
| Glyceryl oleate MB | 6.88 | 7.94 |
| Glycerine | 3.56 | 4.11 |
| Sodium chloride | 2.63 | 3.03 |
| Behentrimonium chloride | 2.00 | 2.30 |
| Isopropyl alcohol | 0.50 | 0.58 |
| Tetrasodium glutamate diacetate | 0.1 | 0.12 |
| Total | 100.00 | 100.00 |

As with Example 1, the resulting solid hair conditioner composition bars were of high quality, perfectly suitable for use, had an acceptable wear rate for effective conditioner performance whilst having sufficient longevity on repeated usage, although bars were softer and their wear rate was higher than the bars of Example 1.

The solid hair conditioner composition bars of Example 7 had good processability and had a similar pH to Example 1.

### Example 8

The solid combination hair shampoo and conditioner composition (2 in 1) of Example 8 was formed from the following ingredients and using the same procedure described above for Example 1. 9 wt % of Mackam LAB 30 was used to provide the 2.7 wt % of lauryl betaine, 5.67 wt % water and 0.63 wt % sodium chloride. SCI was included as the non-soap anionic surfactant.

| | wt % as added | wt % in |
|---|---|---|
| Ingredient | (in emulsion) | product |
| Hydrogenated vegetable oil | 40.00 | 47.29 |
| Water | 25.57 | 12.00 |
| Pureact 1-78 (83-89 wt % SCI) | 20.00 | 23.65 |
| Lauryl betaine | 2.70 | 3.19 |
| Glyceryl oleate MB | 7.00 | 8.28 |
| Glycerine | 3.00 | 3.55 |
| Sodium chloride | 0.63 | 0.74 |
| Behentrimonium chloride | 0.80 | 0.94 |
| Isopropyl alcohol | 0.20 | 0.24 |
| Tetrasodium glutamate diacetate | 0.10 | 0.12 |
| Total | 100.00 | 100.00 |

Pureact 1-78 is a Trade Mark of Innospec Performance Chemicals, of Ellesmere Port, UK. Its major component is sodium cocoyl isethionate (SCI), 83-89 wt %; generally also having <1 wt % water, <14 wt % coconut fatty acid and 4-7 wt % sodium isethionate.

The resulting solid combination hair shampoo and conditioner composition bars were of high quality, perfectly suitable for use, had an acceptable wear rate for effective shampoo and conditioner performance whilst having sufficient longevity on repeated usage, had good processability and had a pH of around 6.

### Example 9

The solid combination hair shampoo and conditioner composition bar of Example 9 was formed from the following ingredients and using the same procedure described above for Example 1. 9 wt % of Mackam LAB 30 was used to provide the 2.7 wt % of lauryl betaine, 5.67 wt % water and 0.63 wt % sodium chloride. SCI was included as the non-soap anionic surfactant.

| | wt % as added | wt % in |
|---|---|---|
| Ingredient | (in emulsion) | product |
| Hydrogenated vegetable oil | 40.00 | 47.29 |
| Water | 25.57 | 12.00 |
| Pureact 1-78 (83-89 wt % SCI) | 20.00 | 23.65 |
| Lauryl betaine | 2.70 | 3.19 |
| Durosoft PG4L | 7.00 | 8.28 |
| Glycerine | 3.00 | 3.55 |
| Sodium chloride | 0.63 | 0.74 |
| Behentrimonium chloride | 0.80 | 0.94 |
| Isopropyl alcohol | 0.20 | 0.24 |
| Tetrasodium glutamate diacetate | 0.10 | 0.12 |
| Total | 100.00 | 100.00 |

The resulting solid combination hair shampoo and conditioner composition bars were of high quality, perfectly suitable for use, had an acceptable wear rate for effective shampoo and conditioner performance whilst having sufficient longevity on repeated usage, had good processability and had a pH of around 6.

### Example 10

The solid hair conditioner bar of Example 10 was formed from the following ingredients and using the same procedure described above for Example 1.

| | wt % as added | wt % in |
|---|---|---|
| Ingredient | (in emulsion) | product |
| Hydrogenated vegetable oil | 57.36 | 64.29 |
| Water | 21.48 | 12.00 |
| Lauryl betaine | 7.80 | 8.74 |
| Glyceryl oleate MB | 6.88 | 7.71 |
| Glycerine | 3.56 | 3.99 |
| Sodium chloride | 1.82 | 2.04 |
| Behentrimonium chloride | 0.80 | 0.90 |
| Isopropyl alcohol | 0.20 | 0.22 |
| Tetrasodium glutamate diacetate | 0.10 | 0.11 |
| Total | 100.00 | 100.00 |

As with Example 1, the resulting solid hair conditioner composition bars were of high quality, perfectly suitable for use, had an acceptable wear rate for effective conditioner performance whilst having sufficient longevity on repeated usage, has good processability and had a similar pH to Example 1.

### Example 11

The solid hair conditioner bar of Example 11 was formed from the following ingredients and using the same procedure described above for Example 1.

| | wt % as added | wt % in |
|---|---|---|
| Ingredient | (in emulsion) | product |
| Stearic acid | 54.36 | 60.96 |
| Water | 21.53 | 12.00 |
| Lauryl betaine | 10.20 | 11.44 |
| Glyceryl oleate MB | 6.88 | 7.72 |
| Glycerine | 3.56 | 3.99 |
| Sodium chloride | 2.38 | 2.67 |
| Behentrimonium chloride | 0.80 | 0.90 |
| Isopropyl alcohol | 0.20 | 0.22 |
| Tetrasodium glutamate diacetate | 0.09 | 0.11 |
| Total | 100.00 | 100.00 |

As with Example 1, the resulting solid hair conditioner composition bars were of high quality, perfectly suitable for use, had an acceptable wear rate for effective conditioner performance whilst having sufficient longevity on repeated usage, has good processability and had a similar pH to Example 1.

### Example 12

The solid hair conditioner bar of Example 12 was formed from the following ingredients and using the procedure described below.

| | wt % as added | wt % in |
|---|---|---|
| Ingredient | (in emulsion) | product |
| Hydrogenated sunflower oil | 54.24 | 63.58 |
| Water | 24.93 | 12.00 |
| Lauryl betaine | 4.50 | 5.28 |
| Glyceryl oleate MB | 8.50 | 9.96 |
| Glycerine | 3.56 | 4.17 |
| Sodium chloride | 1.25 | 1.47 |
| Citric acid | 0.025 | 0.029 |
| Guar HPTM chloride | 1.00 | 1.172 |
| Sodium stearoyl glutamate | 1.00 | 1.172 |
| Sodium cocyl glutamate | 1.00 | 1.172 |
| Total | 100 | 100 |

15 wt % of Mackam LAB 30 was used to provide the 4.50 wt % of lauryl betaine, 9.75 wt % water and 0.75 wt % sodium chloride.

Sodium stearoyl glutamate and sodium cocyl glutamate were included as the non-soap anionic surfactant, both being amino acid-based anionic surfactants. The sodium stearoyl glutamate used was Amisoft HS, available from Ajinomoto OmniChem. The sodium cocyl glutamate used was Amisoft CS, available from Ajinomoto OmniChem.

The sodium chloride used was 2.50 wt % of a 20 wt % aqueous solution of sodium chloride.

The citric acid used was 0.05 wt % of a 50 wt % aqueous solution of citric acid.

The guar HPTM chloride was provided by Jaguar C 162, available from Solvay.

The other ingredients were as noted above.

The total water content of the emulsion was provided by the water present in the Mackam LAB 30, the sodium chloride solution, the citric acid solution and 13.15 wt % added water.

The guar HPTM chloride and glycerine were stirred at room temperature for 30 minutes. 13.15 wt % of water was added and stirring continued for 15 minutes. The sodium chloride and citric acid solutions were then added and the mixture heated to 90° C. The lauryl betaine MB, sodium stearoyl glutamate and sodium cocyl glutamate were added to this mixture and then the hydrogenated sunflower oil and Glyceryl oleate MB were added and heating at 90° C. with recirculation was continued for 30 minutes, to provide the emulsion with the composition shown above.

The emulsion was formed into bars using the method described above for Example 1.

As with Example 1, the resulting solid hair conditioner composition bars were of high quality, perfectly suitable for use, had an acceptable wear rate for effective conditioner performance whilst having sufficient longevity on repeated usage, has good processability and had a similar pH to Example 1.

In summary, the present invention provides a solid hair conditioner composition. The composition comprises the components (a) at least 2 wt % of a non-ionic or amphoteric surfactant; (b) at least 0.1 wt % of a hair conditioning agent; (c) at least 30 wt % of a carrier having a melting point of at least 45° C.; (d) at least 1 wt % of an emulsifier; and (e) at least 3 wt % water; wherein the composition is a solid throughout the range from 0° C. to 40° C. The composition may provide solid hair conditioner bars with good conditioning performance, feel and wear rate, a desired pH and which can be manufactured using conventional soap making methods and equipment. A method of method of manufacturing the solid hair conditioner composition and an emulsion for the preparation of the solid hair conditioner composition are also provided.

Although a few preferred embodiments have been shown and described, it will be appreciated by those skilled in the art that various changes and modifications might be made without departing from the scope of the invention, as defined in the appended claims.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1% by weight of non-specified components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

For the avoidance of doubt, wherein amounts of components in a composition are described in wt %, this means the weight percentage of the specified component in relation to the whole composition referred to.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A solid hair conditioner composition which comprises:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 3 wt % water;
wherein the composition is a solid throughout the range from 0° C. to 40° C.

2. The solid hair conditioner composition according to claim 1, wherein component (a) is a betaine.

3. The solid hair conditioner composition according to claim 1, wherein component (a) provides from 2 to 20 wt % of the solid hair conditioner composition.

4. The solid hair conditioner composition according to claim 1, wherein component (b) is a cationic surfactant.

5. The solid hair conditioner composition according to claim 1, wherein component (b) provides from 0.1 to 5 wt % of the solid hair conditioner composition.

6. The solid hair conditioner composition according to claim 1, wherein component (c) comprises one or more of a wax, a triglyceride, a fatty acid or a fatty alcohol.

7. The solid hair conditioner composition according to claim 1, wherein component (c) provides from 30 to 70 wt % of the solid hair conditioner composition.

8. The solid hair conditioner composition according to claim 1, wherein component (d) has an HLB of from 2 to 6.

9. The solid hair conditioner composition according to claim 1, wherein component (d) is selected from sorbitan fatty acid esters, ethoxylated sorbitan fatty acid esters and glycerol or polyglycerol esters of fatty acids.

10. The solid hair conditioner composition according to claim 1, wherein component (d) provides from 2 to 12 wt % of the solid hair conditioner composition.

11. The solid hair conditioner composition according to claim 1, wherein component (e) provides from 5 to 20 wt % of the solid hair conditioner composition.

12. The solid hair conditioner composition according to claim 1, comprising a component (f) of at least 15 wt % of a non-soap anionic surfactant.

13. The solid hair conditioner composition according to claim 1, wherein the solid hair conditioner composition is in the form of noodles, pellets, flakes or powder.

14. The solid hair conditioner composition according to claim 1, wherein the solid hair conditioner composition is in the form of a block, bar or tablet.

15. The solid hair conditioner composition according to claim 1, wherein the solid hair conditioner composition is soap-free.

16. A method of manufacturing a solid hair conditioner composition, the solid hair conditioner comprising:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 3 wt % water;
wherein the composition is a solid throughout the range from 0° C. to 40° C.;
and wherein the method comprises the steps:
(i) preparing an emulsion containing the components (a) to (e), but with a greater mass of water present than in the solid hair conditioner composition, the temperature of the emulsion being at least 45° C.,
(ii) subjecting the emulsion to cooling and solidification, wherein the water content is reduced during the cooling and solidification to produce the solid hair conditioner composition having components (a) to (e) in the amounts defined.

17. The method according to claim 16, wherein the emulsion at a temperature of at least 45° C. is a free-flowing liquid.

18. The method according to claim 16, wherein the mass of water initially present in the emulsion in step (i) is at least 40% higher than the mass of water present in the solid hair conditioner composition after step (ii).

19. The method as claimed in claim 16, wherein the method comprises a step (iii) of consolidating the solid hair conditioner composition into a block, bar or tablet of the solid hair conditioner composition.

20. An emulsion for the preparation of a solid hair conditioner composition, the emulsion comprising:
(a) at least 2 wt % of a non-ionic or amphoteric surfactant;
(b) at least 0.1 wt % of a hair conditioning agent;
(c) at least 30 wt % of a carrier having a melting point of at least 45° C.;
(d) at least 1 wt % of an emulsifier; and
(e) at least 10 wt % water;
wherein the emulsion is at a temperature of at least 45° C.
